(19) United States
(12) Patent Application Publication
(10) Pub. No.: US 2018/0050018 A1
(43) Pub. Date: Feb. 22, 2018

(54) PERIPHERAL-ANTICHOLINERGIC MUSCARINIC AGONIST COMBINATION

(71) Applicant: Chase Pharmaceuticals Corporation, Parsippany, NJ (US)

(72) Inventors: Kathleen E. CLARENCE-SMITH, Washington, DC (US); Thomas N. CHASE, Washington, DC (US)

(73) Assignee: Chase Pharmaceuticals Corporation, Parsippany, NJ (US)

(21) Appl. No.: 15/556,207

(22) PCT Filed: Mar. 4, 2016
(86) PCT No.: PCT/US16/20837

§ 371 Date: Sep. 6, 2017

Related U.S. Application Data
(60) Provisional application No. 62/129,289, filed on Mar. 6, 2015.
(60) Provisional application No. 62/204,021, filed on Aug. 12, 2015.

(51) Int. Cl. A61K 31/4015; A61K 31/435; A61K 31/439; A61K 45/06; C07D 471/10; C07D 491/10

(52) U.S. Cl. CPC A61K 31/4015; A61K 31/435; C07D 491/10; A61K 45/06; C07D 471/10; A61K 31/439

(57) ABSTRACT

A combination of a non-selective, peripheral anticholinergic agent, and a muscarinic receptor agonist, optionally with an acetyl cholinesterase inhibitor, and method of using the same for the treatment of hypocholinergic disorders of the central nervous system. The combination of the present invention allows for safe administration of high doses of muscarinic receptor agonist, and improved efficacy of the muscarinic receptor agonist for treatment of hypocholinergic disorders of the central nervous system. The combination also allows for a maximum supply of acetylcholine to the central nervous system, when an acetyl cholinesterase inhibitor is used in combination with a non-selective, peripheral anticholinergic agent and a muscarinic receptor agonist.

### CROSS-REFERENCE TO RELATED APPLICATIONS

This application claims benefit of U.S. Provisional Application No 62/129,289, filed Mar. 6, 2015; and U.S. Provisional Application No. 62/204,021, filed Aug. 12, 2015; the entire disclosures of each of which are hereby incorporated herein by reference.

### FIELD OF THE INVENTION

The invention pertains to the field of the treatment of hypocholinergic disorders of the central nervous system, in particular of Alzheimer's Disease (AD), Alzheimer type dementia, AD-type dementia, Parkinson's dementia, Lewy body diseases, schizophrenia, and chronic neuropathic pain; and proposes a new combination of an agonist and of an antagonist of the same receptor. More particularly, the invention proposes a combination of a muscarinic antagonist consisting of a non-selective, peripheral muscarinic receptor antagonist having anticholinergic activity, herein below referred to as non-selective Peripheral Anti-Cholinergic Agent (“nsPAChA”) and of a muscarinic agonist consisting of a Cholinergic Receptor Agonist (CRA).

### DEFINITIONS

“CNS”: Central Nervous System.
“PNS”: Peripheral Nervous System.
“AChR”: Acetylcholine Receptor.
“Muscarinic type receptors (mAChRs)”: Five subtypes of muscarinic receptors, M1 through M5, have been identified.
“CRA”: Cholinergic Receptor Agonist acting on the mAChRs, including orthosteric activators and allosteric activators, in particular both allosteric agonists and positive allosteric modulators, of mAChR subtypes.
“nsPAChA(s)”: non-selective, peripheral AntiCholinergic Agent(s) acting on the AChRs which are present in the PNS.
“Non-selective”: refers to nsPAChAs, and applies to muscarinic anticholinergic agents exhibiting inhibitory activity on the mAChRs broadly across the various subtypes of muscarinic M-receptors, namely the M1 -M5 receptors.
“Peripheral”: refers to muscarinic anticholinergic agents and applies to anticholinergics that are largely unable (have a limited ability) to enter the central nervous system following systemic administration and thus do not affect brain function to a clinically appreciable degree. These drugs can include both quaternary and tertiary ammonium anticholinergic agents, especially those having low lipid solubility.
“Anticholinergic therapy”: the treatment with an anticholinergic agent of such medical conditions as gastro-intestinal cramping, nausea, retching, vomiting, fecal incontinence, bladder spasms, urinary incontinence, overactive bladder, asthma, motion sickness, muscular spasms, and smooth muscle contractive disorders; or the treatment, if any, with an anticholinergic agent of side effects caused by CRAs, including, but not limited to gastro-intestinal cramping, nausea, retching, vomiting, fecal incontinence, bladder spasms, urinary incontinence, overactive bladder, asthma, motion sickness, muscular spasms, and smooth muscle contractive disorders.
“CSF”: Cerebrospinal Fluid.
“IR”: Immediate Release of the active ingredient from a composition.
“ER”: Extended Release (or sustained or controlled release) of the active ingredient from a composition by any administration route, in particular oral, parenteral transcutaneous, or transdermal route.
“AChE”: Acetyl Choline esterase
“AChEI(s)”: Acetyl Choline Esterase Inhibitor(s).
“Transdermal delivery” of drug can be targeted to skin tissues just under the skin, regional tissues or organs under the skin, systemic circulation, and/or the central nervous system.
“Transdermal Therapeutic System” (TTS) is targeted to delivery of drug to skin tissues just under the skin, regional tissues, using transdermal drug formulations and transdermal patches incorporating such transdermal drug formulations.

### BACKGROUND OF THE INVENTION

Reduced levels of neurotransmitters including acetylcholine occur in dementias of the Alzheimer type. In particular, a deficit in acetylcholine-mediated transmission is thought to contribute to the cognitive and neurobehavioral abnormalities associated with these disorders. Accordingly, drugs known to augment cholinergic transmission in the CNS are the mainstay of current therapy. In addition, other diseases of the nervous system also involve decreased cholinergic transmission and are referred to as “hypocholinergic syndromes of the nervous system”. Besides AD, and AD-type dementia, such diseases include, but are not limited to, Mild Cognitive Impairment (MCI), Lewy Body Disease dementia (LBD), Parkinson disease dementia (PDD), post-stroke dementia, vascular dementia, Traumatic Brain Injury, Anorexia Nervosa, Down's syndrome, Tourette disease, tardive dyskinesia, Pick's disease, Huntington's chorea, Friedrich's ataxia, chronic neuropathic pain and schizophrenia. It is well documented that schizophrenic patients experience cognitive disturbances that are not well addressed by current medications (reviewed in Foster et al, 2014). CRAs have been reported to dose-dependently improve the cognitive disturbances associated with schizophrenia, but the effect of CRAs is of limited size and dose-dependent side effects prevent further increases in the CRA doses.

Acetylcholinesterase inhibitors (AChEIs) are now not only part of the standard of care for patients suffering from a dementia of the Alzheimer type, but are also widely used off-label for various other chronic progressive hypocholinergic disorders of the nervous system. AChEIs have the enhancement of acetylcholine-mediated neurotransmission as a general mechanism of action. All act in the human CNS to increase and prolong the availability of acetylcholine by inhibiting its degradatory enzyme acetylcholinesterase (AChE). Four AChEIs have been approved by the U.S. FDA for the treatment of dementias of the Alzheimer type: tacrine, donepezil [Aricept®], rivastigmine [Exelon ] and galantamine [Razadyne]. Rivastigmine has also been approved for the treatment of Parkinson's disease dementia. AChEIs are available in various formulations including immediate release forms such as tablets, capsules and solutions as well as rapid dissolving and extended release forms for oral administration as well as those for parenteral (e.g. transdermal) administration.

Unfortunately, however, none of the currently available AChEIs offers more than modest clinical benefit for patients suffering from any of the aforementioned dementing disorders, even when these medications are administered at their maximum safe and tolerated doses. This is the first problem limiting the success of current AChEI therapy of Alzheimer type dementias.

A second problem limiting the success of current AChEI therapy of Alzheimer type dementias is that, even at recommended amounts, all these drugs produce dose limiting adverse reactions, mainly if not exclusively, by over-stimulating peripheral cholinergic receptors of the muscarinic type. As a result, signs and symptoms of untoward gastrointestinal, pulmonary, cardiovascular, urinary, and other systems dysfunction occur. These side effects commonly include, anorexia, nausea, vomiting, diarrhea, abdominal pain, weight loss; increased bronchial secretions, dyspnea, bronchoconstriction and bronchospasm; bradycardia, supraventricular cardiac conduction abnormalities, vasodilation, hypotension, dizziness and syncope; urinary bladder spasm, increased urinary frequency, and incontinence; flushing and diaphoresis; fatigue, headache, lacrymation, miosis, and loss of binocular vision (Physicians' Desk Reference 2008, Thomson PDR, Montvale, N.J.).

These problems linked to the use of the AChEIs have been solved by combining said AChEI with a nsPAChA (U.S. Pat. No. 8,404,701, the disclosure of which is incorporated herein by reference in its entirety) or with a non-anticholinergic antiemetic agent (U.S. Pat. No. 8,877,768, the disclosure of which is incorporated herein by reference in its entirety), these combinations allowing a great increase of the administered AChEI dosage amounts with attending increase in plasma and brain concentrations of the AChEI, and consequent possibility of increasing anti-dementia efficacy.

Another way to increase the cholinergic transmission in the brain is to stimulate post-synaptic cholinergic receptors by administering an agonist of the muscarinic receptors, but the results were generally disappointing.

In fact, many CRAs have been studied in the last two decades but, except for cevimeline (EVOXAC®), which is marketed in the U.S.A. for the limited indication of the treatment of symptoms of dry mouth in patients with Sjögren's Syndrome, none of said CRAs showed a significant activity on the CNS which could be used for the treatment of Alzheimer type dementia or of central hypocholinergic disorders.

The (E)-N-methoxy-1-(1-methyl-1,2,5,6-tetrahydropyridin-3-yl)methanimine, a non-selective muscarinic acetylcholine receptor partial agonist with cognition-acting properties known as milameline and disclosed in U.S. Pat. No. 6,037,347, the disclosure of which is incorporated herein by reference in its entirety, was investigated for the treatment of Alzheimer's disease, but the drug, while possessing a pharmacological profile consistent with that of a muscarinic partial agonist, its central cholinergic action was produced in rats and monkeys at doses slightly lower than those stimulating peripheral cholinergic receptors (Schwarz R D, Callahan M J, Coughenour L L, Dickerson M R, Kinsora J J, Lipinski W J, Raby C A, Spencer C J, Tecle: “Milameline (CI-979/RU35926): a muscarinic receptor agonist with cognition-activating properties: biochemical and in vivo characterization”; J Pharmacol Exp Ther. 1999 November; 291(2):812-22-Schwarz 1999, the disclosure of which is incorporated herein by reference in its entirety). The development of milameline seems to be discontinued.

Similarly, the (3R)-N-methoxyquinuclidine-3-carboximidoyl cyanide hydrochloride known as sabcomeline and disclosed in U.S. Pat. No, 5,278,170, the disclosure of which is incorporated herein by reference in its entirety, is a selective M1 receptor partial agonist that was under development for the treatment of Alzheimer's disease (Loudon J M, Bromidge S M, Brown F, et al.: “SB 202026: a novel muscarinic partial agonist with functional selectivity for Ml receptors”; J Pharmacol Exp Ther. 1997 December; 283(3):1059-68—Louden 1997, the disclosure of which is incorporated herein by reference in its entirety). It was submitted to phase III clinical trials before being discontinued (R & D Focus Drug News, Mar. 8, 2004).

Another CRA, the 5-[4-(hexylsulfanyl)-1,2,5-thiadiazol-3-yl]-1-methyl-1,2,3,6-tetrahydropyridine, known as tazomeline and disclosed in U.S. Pat. No. 5,041,455, the disclosure of which is incorporated herein by reference in its entirety, is a drug which acts as a non-selective muscarinic acetylcholine receptor agonist. It was in clinical trials for the treatment of cognitive dysfunction such as that seen in Alzheimer's disease and schizophrenia, but, according to Wikipedia, its “development was apparently scrapped for unknown reasons” and no sign of an effective development is known.

A close analog of tazomeline, the 3-(4-hexyloxy-1,2,5-thiadiazol-3-yl)-1-methyl-5,6-dihydro-2H-pyridine known as xanomeline and disclosed in U.S. Pat. No. 5,043,345, the disclosure of which is incorporated herein by reference in its entirety, has been disclosed as a muscarinic acetylcholine receptor agonist with reasonable selectivity for the M1 and M4 subtypes. The efficacy of xanomeline, that stimulates muscarinic receptors in the brain and in the periphery was studied in patients with Alzheimer disease in a 6-month double-blind, placebo-controlled, parallel group trial. Compared to placebo, xanomeline was shown to significantly improve cognitive and behavioral symptoms of Alzheimer disease (Bodick et al, 1997), but also caused dose-dependent unacceptable side effects, including bradycardia, gastro-intestinal distress, excessive salivation, and sweating. Such side effects prevented the use of doses of xanomeline that could achieve maximum anti-dementia efficacy and reflect stimulation of cholinergic receptors outside the brain.

Xanomeline is also described in a transdermally administrable form in U.S. Pat. No. 5,980,933, the disclosure of which is incorporated herein by reference in its entirety, and clinical experimentation on said preparation was announced. The paper Mirza 2003 (Mirza et al. CNS Drug Reviews Vol. 9, No. 2, pp. 159-186) confirmed a phase II clinical trial with transdermal xanomeline, but no specific result appeared in the literature after that date.

A xanomeline fluorinated analog, the 3-[3-(3-(3-fluorophenyl)-2-propyn-1-ylthio)-1,2,5-thiadiazol-4-yl]-1,2,5,6-tetrahydro-1 -methylpyridine oxalate, known as EUK 1001, was disclosed by Xiaoping Lei in CN1821243B and considered a promising therapeutic agent for the treatment of AD and age-related memory disorders (Yihui Cui, Dong Wang, Wen Si, Wen Lv, Yan Niu, Xiaoping Lei, Yinhe Hu and Xiaohua Cao: “Enhancement of memory function in aged mice by a novel derivative of xanomeline”; Cell Research; 2008; 18:1151-1153 published online 21 Oct. 2008—Yihui Cui 2008 the disclosure of which is incorporated herein by reference in its entirety). However, no result of clinical trials in human being using EUK1001 appeared in the literature.

Dose-limiting adverse events attending the use of drugs that stimulate cholinergic transmission, such as xanomeline, appear to primarily reflect the excessive stimulation of peripheral cholinergic receptors, especially those of the muscarinic type (mAChRs), such that in both healthy volunteers and Alzheimer's patients many of these side effects have been reported for xanomeline; in the patient population this led to a discontinuation rate higher than 50% while the effects on cognition were not as robust and mainly seen at the highest doses tested (Mirza et al., CNS Drug Reviews Vol. 9, No. 2, pp. 159-186 (2003).

In conclusion, the development of all of the above CRAs was discontinued because the results of the studies were disappointing not for a basic muscarinic inactivity of the products but because said products were inefficacious in patients and, in addition, induced dose-limiting, irreducible adverse effects.

In a review published in NEUROLOGY, 49, July 1997,by H. Robert Brashear, MD, of the book “Muscarinic Agonists and the Treatment of Alzheimer Disease” (Edited by Abraham Fisher—R. G. Landes ,1996), the reviewer concluded his comment as follows: “It will be of interest to most clinicians who treat Alzheimer's disease and valuable to chemical researchers, basic neuroscientists, biochemists, and pharmacologists investigating cholinergic dysfunction and therapy”. Despite this clear interest and the extensive studies made on a series of compounds during the last two decades, none of the studied compounds became a drug for the treatment this disease for the reasons set forth above.

In addition, CRAs consisting of allosteric modulators of the M₁-muscarinic acetylcholine receptor are extensively studied and are the object of copious patent and scientific literature.

A review by B. J. Melancon, J. C Tarr, J. D. Panarese, M. R. Wood and C. W. Lindsley published in Drug Discovery Today; Volume 18, Numbers 23/24, December 2013, “Allosteric modulation of the M₁ muscarinic receptor: improving cognition and a potential treatment for schizophrenia and Alzheimer's disease” (Melancon et al.), the disclosure of which is incorporated herein by reference in its entirety, illustrates the role of the M₁ receptor in Alzheimer's disease and in schizophrenia by referring to selected allosteric modulators of the M₁ receptor.

This review also reports that positive allosteric modulator MK-7622 entered Phase II clinical trials as an adjunct therapy to AChEIs in patients with AD. This positive allosteric modulator of the M₁ receptor, 3-[(1S,2S)-2-hydroxycyclohexyl]-6-[(6-methylpyridin-3-yl)methyl]benzo[h]quinazolin-4(3H)-one, is described in U.S. Pat. No. 8,883,810, the disclosure of which is incorporated herein by reference in its entirety.

Notwithstanding the previous aforementioned disappointing results and the last progress of the scientific studies, the literature does not teach how to take advantage of the ubiquitous, potent activity of the muscarinic agonists safely. Thus, the problem of the effective and safe treatment of Alzheimer type dementia and in general of hypocholinergic disorders in the CNS such as schizophrenia, Down's syndrome, Tourette disease, tardive dyskinesia, Picks disease, Huntington's chorea, and Friedrich's ataxia, with a muscarinic agonist, remains of primary importance. Until the present invention, no means of taking advantage of the potent cholinergic activity of a muscarinic agonist for the treatment of the above diseases had been discovered.

An improvement in the treatment of Alzheimer type dementia is attained by a combined therapy associating a non-selective, peripheral anticholinergic agent, at a dose of from 20% to 200% the current daily doses, with an AChEI, at a dose up to about 6 times the maximal recommended dose of said AChEI, as disclosed in U.S. Pat. No. 8,404,701, the disclosure of which is herein incorporated by reference in its entirety. By such a treatment, a higher acetylcholinesterase inhibition in the CNS is achieved and greater relief of the symptoms of Alzheimer type dementia is enabled, by concomitantly decreasing concurrent adverse effects. This result was obtained by successfully inferring that the good dose-response obtained with the AChEIs, i.e. with enzyme inhibitors, would allow an increase of the inhibition of AChE in the CNS with a safe increase of the AChEI dose. Conversely, in the case of the muscarinic receptors, nothing in the literature suggests how to modulate the product dose in the presence of five muscarinic receptors and of the intrinsic properties of the CRAs. In particular, the literature does not give any indication or suggestion for exploiting the potential of said muscarinic agonists.

U.S. Pat. No. 8,877,768, the disclosure of which is herein incorporated by reference in its entirety, discloses an improvement in the treatment of Alzheimer type dementia, which is attained by a combined therapy associating a non-anticholinergic-antiemetic agent, at a dose of from 50% to 300% the current IR daily doses, with an AChEI, at a dose up to 4 times the maximal recommended doses of said AChEI when administered alone. However, the used antiemetics, which are non-anticholinergic by definition, do not interfere with both the central and peripheral activity of the AChEIs.

As mentioned above, a copious patent literature discloses the CRAs in the field of the present invention, including the allosteric modulators of the M₁ receptor, alone or in combination with other drugs.

For example U.S. Pat. No. 8,883,810 (see also WO 2010/059773), describing the MK-7622, cites the combination of a class of aryl methyl benzoquinazolinone compounds disclosed therein with other drugs to render the administration safer or more effective or to reduce the risk of side effects or toxicity of said aryl methyl benzoquinazolinones. These combinations include anticholinergic drugs but the document does not disclose any non-selective, peripheral anticholinergic drug. On the contrary, it specifically cites biperiden and trihexyphenidyl hydrochloride as anticholinergics, both being central anticholinergic agents for the treatment of the Parkinson's disease.

US2011/0020423 discloses the combination of one or more muscarinic “Activators” (e.g., agonist, partial agonist, co-agonist, physiological agonist, potentiator, stimulator, allosteric potentiator, positive allosteric modulator or allosteric agonist) and one or more muscarinic “Inhibitors” (e.g., antagonist, partial antagonist, competitive antagonist, non-competitive antagonist, uncompetitive antagonist, silent antagonist, inverse agonist, reversible antagonist, physiological antagonist, irreversible antagonist, inhibitor, reversible inhibitor, irreversible inhibitor, negative allosteric modulator, or allosteric antagonist).

U.S. Pat. No. 8,853,219 discloses muscarinic agonists, which are useful for stimulating muscarinic receptors and treating cognitive disorders, said agonists including oxadiazole and oxathiazole derivatives, in particular 5-(3-ethyl-1,2,4-oxadiazol-5-yl)-1,4,5,6-tetrahydropyrimidine, also known as MCD-386, which is described in the literature for example in U.S. Pat. No. 5,403,845 to Dunbar, et al., 3-Methyl-5-(piperidin-3-yl)-1,2,4-oxadiazole), as a racemic mixture and as the single stereoisomers. This document also discloses combination compositions and co-administration comprising muscarinic agonists and antagonists, said muscarinic agonists including the substituted oxadiazoles and thiadiazoles disclosed therein and said muscarinic antagonists including atropine sulfate, N-methylatropine nitrate, flavoxate hydrochloride, N-methylscopolamine hydrochloride (methscopolamine), oxybutynin chloride, glycopyrrolate bromide, darifenacin hydrobromide, solifenacin succinate, propantheline bromide, trospium chloride, tolterodine tartrate, fesoterodine fumarate, methantheline bromide and combinations thereof.

In terms of co-administration of a muscarinic-antimuscarinic combination, this document intends separate administration of agonist and antagonist, e.g., in separate dosage forms such as separate pills, separate injectable solutions or separate iontophoretic patches. According to this document, pharmacological tests made with a combination of representative oxadiazole muscarinic agonists with muscarinic antagonists showed that darifenacin and oxybutynin, both tertiary amines, are less effective than the other muscarinic antagonists by both oral and iontophoretic patch administration. In addition, this document observes that these drugs are known to penetrate the blood-brain barrier and may therefore inhibit the therapeutic effects of the agonist in the brain. Thus, this document, does not make any distinction among the peripheral/non-peripheral and selective/non-selective antimuscarinic agents.

In summary, notwithstanding great scientific effort, the problem of the safe treatment of other hypocholinergic disorders of the nervous system such as Parkinson's dementia, Lewy body diseases, Down Syndrome, and chronic neuropathic pain remains unsolved.

### SUMMARY OF THE INVENTION

It has now been found that an nsPAChA, when concurrently or sequentially administered in combination with a CRA, is able to neutralize the adverse effects that hindered the development of a muscarinic agonist for the treatment of central disorders due to a deficit of acetylcholine in the brain and to allow the modulation of the CRA's doses in order to optimize the response of the patient to the cholinergic treatment. In fact, by treating a human with an nsPAChA, it is possible to safely administer a CRA, even at high doses thus, in case of a patient suffering from Alzheimer type dementia, allowing said CRA to safely activate the acetylcholine receptors and to improve cognition.

In particular, by treating a human with a nsPAChA, it is possible to safely administer even high doses of a CRA to a patient suffering from hypocholinergic disorders of the central nervous system, such as AD, AD-type, Mild Cognitive Impairment (MCI), Lewy Body Disease dementia (LBD), Parkinson disease dementia (PDD), post-stroke dementia, vascular dementia, Traumatic Brain Injury, Down syndrome, Anorexia Nervosa, Down's syndrome, Tourette disease, tardive dyskinesia, Pick's disease, Huntington's chorea, Friedrich's ataxia, chronic neuropathic pain and schizophrenia, thus allowing said CRA to safely activate the acetylcholine receptors and to improve cognition.

The finding of the present invention was unexpected in view of the disclosures of the prior art, in particular in view of the knowledge of, one side, the lack of efficacy of the muscarinic cholinergic receptor agonists at the doses administered to the patients and, on the other side, of the irreducible adverse effects induced by said agonists at said administered doses. On the contrary, it has been found that the administration of a CRA concurrently with a nsPAChA, will not produce any adverse effect not only at the CRA doses normally administered to a human, but also at doses which would otherwise be unquestionably intolerable for said human.

In particular, this finding eliminates the dose-limit that, in the past, caused the failure of all the clinical trials, thus providing a new tool for treating Alzheimer type dementia and in general central hypocholinergic disorders of the CNS by enabling the full efficacy of CRAs. Said new tool comprises treating a patient in need of such a treatment with a high dose of an nsPAChA, in combination with a CRA. This treatment occurs, on one hand without the onset of CRA-associated peripheral dose-limiting adverse effects and, on the other hand, without the onset of nsPAChA central adverse effects, because these anticholinergics are substantially peripheral.

Thus, the present invention provides a combination of an nsPAChA and of a CRA which is useful for the treatment of Alzheimer type dementia and for CNS hypocholinergic disorders. More particularly, in said combination said CRA is used at a dose that would have been intolerable in the absence of said nsPAChA. In practice, said CRA may be present in said combination at a dose that is higher than the mean maximal tolerated dose which was determined during the clinical trials.

In addition, the present invention provides the combination of an nsPAChA with a CRA, said combination being formulated in the same unit form.

Finally, the present invention also provides the addition of an AChEl to the above nsPAChA/CRA combination, thus assuring a maximum supply of acetylcholine to the CNS.

### DETAILED DESCRIPTION

The present invention provides a pharmaceutical combination comprising as Components:

(a) a muscarinic receptor antagonist selected from the group consisting of the non-selective, peripheral anticholinergic agents (nsPAChAs); and
(b) a muscarinic receptor agonist selected from the group consisting of cholinergic receptor agonists (CRA).

This combination may be used for the treatment of Alzheimer type dementia and more generally for hypocholinergic disorders of the central nervous system, including Parkinson's disease dementia, Lewy Body Dementia, Frontotemporal Lobar Dementia, Mild Cognitive Impairment (MCI), Vascular Dementia, Traumatic Brain Injury, Down's Syndrome, Anorexia nervosa, and Schizophrenia.

### The nsPAChAs

The nsPAChAs used as Component (a) are quaternary ammonium nsPAChAs, sulfonium nsPAChAs, (1S)-(3R)-1-azabicyclo [2.2.2] oct-3-yl 3,4-dihydro-1-phenyl-2(1H)-iso-quinolinecarboxylate (solifenacin) and pharmaceutically acceptable salts and solvates thereof, 1-methylpiperidin-4-yl) 2,2-di(phenyl)-2-propoxyacetate (propiverine) and pharmaceutically acceptable salts and solvates thereof, 1,4,5,6-tetrahydro-1-methylpyrimidin-2-ylmethyl α-cyclohexyl-α-hydroxy-α-phenylacetate (oxyphencyclimine) and pharmaceutically acceptable salts and solvates thereof, (R)-N,N-diisopropyl-3-(2-hydroxy-5-methylphenyl)-3-phenylpropanamine (tolterodine) and pharmaceutically acceptable salts and solvates thereof, [2-[(1R)-3-(di(propan-2-amino)-1-phenylpropyl]-4-(hydroxymethyl)phenyl] 2-methylpropanoate (fesoterodine) and pharmaceutically acceptable salts and solvates thereof

Said nsPAChAs, preferably, are compounds with a duration of action of at least 6 hours, advantageously from 8 to 24 hours, more advantageously from 10 to 24 hours, preferably from 12 to 24 hours, even though nsPAChAs having an appropriate duration of action corresponding to the duration of action of the concomitantly administered CRA may be successfully used.

Typical quaternary ammonium nsPAChAs or sulfonium nsPAChAs are compounds of formula I

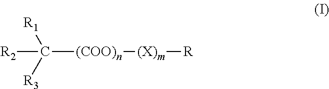

wherein
R is a radical selected from the group consisting of those of formulas (a)-(e)

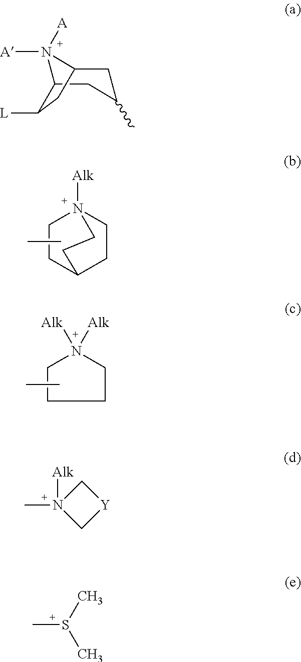

A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group.

Exemplary nsPAChAs of formula I above useful for the treatment of Alzheimer type dementia in combination with CRAs are
anisotropine methylbromide [R=(a), A=A′=CH₃, L=H; n=1; m=0; R₁═R₂=n-C₃H₇; R₃═H;];
ciclotropium bromide [R=(a), A=CH₃, A′=isopropyl, L=H; n=1; m=0; R₁=phenyl; R₂=cyclopentyl; R₃═H];
flutropium bromide [R=(a), A=CH₃, A′=2-fluoroethyl, L=H; n=1; m=0; R₁═R₂=phenyl; R₃═OH];
homatropine methyl bromide [R =(a), A=A′=CH₃, L=H; n=1; m=0; R₁=phenyl; R₂═R₃═H];
sintropium bromide; [R=(a), A=CH₃, A′=isopropyl, L=H; n=1; m =0; R₁=R₂=n-C₃H₇; R₃═H];
tematropium metilsulfate [R =(a), A=A'=CH₃, L=H; n=1; m=0; R₁=phenyl; R₂═COOC₂H₅; R₃═H]; tropenziline bromide [R=(a), A=A′=CH₃, L=methoxy; n=1; m=0;R₁═R₂=phenyl, R₃═OH];
trospium chloride [R=(a), A+A′=1,4-butylene, L=H; n=1; m=0; R₁═R₂=phenyl; R₃═OH];
clidinium bromide [R=(b)-3-, Alk=methyl; n=1; m=0; R₁=R₂=phenyl; R₃═OH];
droclidinium bromide [R=(b)-3-, Alk=methyl; n=1; m=0; R₁=phenyl; R₂=cyclopentyl; R₃═OH];
benzilonium bromide [R=(c)-3-, both Alk and Alk′=ethyl; n=1; m=0; R₁═R₂=phenyl; R₃═OH];
benzopyrronium bromide [R=(c)-3-, both Alk and Alk′=methyl; n=1; m=0; R₁═R₂=phenyl; R₃═OH];
cyclopyrronium bromide [R=(c)-3-, Alk=methyl and Alk′=ethyl; n=1; m=0; R₁=phenyl; R₂=cyclopentyl; R₃═H];
glycopyrronium bromide (glycopyrrolate) [R=(c)-3-, both Alk and Alk′=methyl; n=1; m=0; R₁=phenyl; R₂=cyclopentyl; R₃═H];
heteronium bromide [R=(c)-3-, both Alk and Alk′=methyl n=1; m=0; R₁=phenyl; R₂=2-thienyl; R₃═OH];
hexopyrronium bromide [R=(c)-3-, both Alk and Alk′=methyl; n=1; m=0; R₁=phenyl; R₂=cyclohexyl; R₃═H];
oxypyrronium bromide [R=(c)-2-, both Alk and Alk′=methyl; n=1; m=1; X=1,2-ethylene; R₁=phenyl; R₂=cyclohexyl; R₃═OH];
ritropirronium bromide [R=(c)-3-, both Alk and Alk′=methyl; n=1; m=0; R₁=phenyl; R₂=cyclopentyl; R₃═OH];
etipirium iodide [R=(d), Alk=methyl, Y=1,2-ethylene; n=1; m=1; X=1,2-ethylene; R₁═R₂=phenyl; R₃═OH];
fenclexonium methylsulfate [R=(d), Alk=CH₃, Y=1,3-propylene; n=0; m=1; X=1,2-ethylene; R₁=phenyl; R₂=1-cyclohexenyl ; R₃═H];
tricyclamol chloride (procyclidine methochloride) [R=(d), Alk=methyl, Y=1,2-ethylene; n=0; m=1; X=1,2-ethylene; R₁=phenyl; R₂=cyclohexyl; R₃═OH];
tiemonium iodide [R=(d), Alk=methyl, Y=2-oxa-1,3-propylene; n=0; m=1; X=1,2-ethylene; R₁=phenyl; R₂=2-thienyl; R₃═OH];
hexasonium iodide [R=(e); n=1; m=1; X=1,2-ethylene; R₁=phenyl; R₂=cyclohexyl; R₃═H]; and
oxysonium iodide [R=(e); n=1; m=1; X=1,2-ethylene ; R₁=phenyl; R₂=cyclohexyl; R₃═OH.

Other typical, commercial nsPAChAs, not included in Formula I above, are scopolamine methobromide, scopolamine butylbromide, scopolamine methonitrate, isopropamide iodide, valethamate bromide, atropine methobromide, atropine methonitrate, diponium bromide, pipenzolate bromide, penthienate bromide, benactizine methobromide, diphemanil, emeprioum bromide and dibutoline sulfate.

Advantageous nsPAChAs are the tertiary or quaternary compounds available in drugs for current anticholinergic therapy, in particular anisotropine hydrobromide, available with a maximum dose/unit form of 100 mg; butylscopolamine bromide, with a maximum dose/unit form of 10 mg; cimetropium bromide, with a maximum dose/unit form of 50 mg; clidinium bromide, with a maximum dose/unit form of 2.5 mg; ER fesoterodine fumarate, with a maximum dose/unit form of 8 mg; glycopyrronium bromide, with a maximum dose/unit form of 2 mg; otilonium bromide, with a maximum dose/unit form of 40 mg; prifinium bromide, with a maximum dose/unit form of 30 mg; IR propiverine hydrochloride, with a maximum dose/unit form of 15 mg; ER propiverine hydrochloride, with a maximum dose/unit form of 30 mg; solifenacin succinate, with a maximum dose/unit form of 10 mg; timepidium bromide, with a maximum dose/unit form of 30 mg; IR trospium chloride, with a maximum dose/unit form of 20 mg; ER trospium chloride, with a maximum dose/unit form of 60 mg; and valethamate bromide, with a maximum dose/unit form of 10 mg.

Azoniaspiro [3β-benziloyloxy-(1α,5α)-nortropane-8,1′-pyrrolidine] chloride (formula I, A+A′=1,4-butylene) described in U.S. Pat. No. 3,480,626, known under its International Non-proprietary Name trospium chloride; the tartrate, maleate, fumarate and succinate salts of trospium; solifenacin, described in U.S. Pat. No. 6,017,927, and the compound thereof with succinic acid; propiverine hydrochloride, described in DD 106643, and its quaternary methylpropiverinium iodide and methylpropiverium bromide, described in WO 2014/025569; oxyphencyclimine, described in GB 795758, and the hydrochloride thereof; tolterodine, described in U.S. Pat. No. 5,382,600, and the hydrogen tartrate thereof; fesoterodine, described in U.S. Pat. No. 5,382,600, and the fumarate thereof, are the preferred nsPAChAs. Other pharmaceutical acceptable salts of trospium, in particular those with succinic acid and tartaric acid, are cited in US 2006/0293356.

Glycopyrronium bromide; trospium chloride, which is a long-acting nsPAChA whose absorbed amount, even though poor, has an average plasma half-life of about 18 hours; solifenacin succinate, which also has a long half-life; propiverine hydrochloride and the aforementioned quaternary ammonium salts thereof, are particularly preferred.

For the intended use, the nsPAChA Component (a) is formulated in pharmaceutical compositions comprising, as an active ingredient thereof, said nsPAChA in admixture with a pharmaceutical carrier.

Said Component (a) is present in an amount that allows the reduction of peripherally mediated adverse effects that would be caused by the administration of doses of CRA which are higher that the maximal tolerated dose found for each of them in the clinical trials of said CRA.

In a preferred embodiment, the amount of an nsPAChA that is commercially available for the anticholinergic therapy, such as the aforementioned tertiary and quaternary nsPAChAs, is from 0.5 to 6 times the maximum amount contained in the IR-forms of the marketed drugs. More particularly, according to this preferred embodiment the nsPAChA amount in a compositions as IR-formulation is from 0.5 to 4 times, preferably from 1.2 to 4 times the maximum amount contained in the marketed drugs in IR form and the nsPAChA amount in a compositions as ER-formulation is from 0.75- to 6-times, preferably from 1.2- to 6-times the maximum amount contained in the marketed drugs in IR form.

Thus, the present invention also provides a pharmaceutical composition in an

IR-form comprising, as an active ingredient, a nsPAChA selected from the group consisting of
anisotropine hydrobromide, in an amount of from 120 mg to 400 mg;
butylscopolamine bromide, in an amount of from 12 mg to 40 mg;
cimetropium bromide, in an amount of from 55 mg to 200 mg;
clidinium bromide, in an amount of from 3 mg to 10 mg;
fesoterodine fumarate ER, in an amount of from 9.6 mg to 32 mg;
glycopyrronium bromide, in an amount of from 2.4 mg to 8 mg;
otilonium bromide, in an amount of from 48 mg to 160 mg;
oxyphencyclimine, in an amount of from 18 mg to 60 mg;
prifinium bromide, in an amount of from 36 mg to 120 mg;
propiverine hydrochloride IR, in an amount of from 18 mg to 60 mg;
propiverine hydrochloride ER, in an amount of from 36 mg to 120 mg;
solifenacin succinate, in an amount of from 12 mg to 40 mg, normally from 12 mg to 20 mg;
tolterodine hydrogen tartrate, in an amount of from 4.8 mg to 16 mg;
timepidium bromide, in an amount of from 36 mg to 120 mg;
trospium chloride IR, in an amount of from 24 mg to 80 mg;
trospium chloride ER, in an amount of from 72 mg to 240 mg; and
valethamate bromide, in an amount of from 12 mg to 40 mg;
   in admixture with a pharmaceutical carrier.

The compositions prepared using the nsPAChAs according to the present invention allow the administration of 1.2- to 4-times and even 1.2- to 6-times the maximal tolerated dose of CRA, as averagely determined in the clinical trials, to patients suffering of Alzheimer type dementia, without clinically significant symptoms of peripheral cholinergic system overstimulation.

The compositions are preferably formulated in dosage unit forms for oral or parenteral, in particular transdermal, administration, wherein the active ingredient is mixed with a pharmaceutical carrier.

The pharmaceutical compositions prepared using the nsPAChAs Component (a) according to the present invention are indicated in the treatment of the symptoms of Alzheimer type dementias in combination with even high doses of a CRA Component (b), concurrently or sequentially administered therewith, in order to improve to a greater extent said symptoms without adverse effects.

Thus, the invention provides a method for treating Alzheimer type dementia, which comprises administering to a patient in need of said treatment the above-illustrated combination. In such a treatment, Component (a) and Component (b) of the combination may be administered simultaneously or sequentially to said patient, Compound (a) being indifferently administered before or after Compound (b). Compounds (a) and/or (b) may also be administered by the same or a different administration route.

The invention also provides the use of a third component, Component (c), consisting of an AChEI, also formulated in a pharmaceutical composition.

According to an advantageous embodiment, the pharmaceutical compositions prepared by using the nsPAChAs according to the present invention are present in unit forms also containing a CRA that acts as direct cholinergic agonist in the CNS to improve the symptoms of Alzheimer type dementia, in a quantity sufficient to maximally alleviate disease-associated neurobehavioral symptoms, with minimum of treatment-associated adverse effects.

By using the combination of the invention, it is possible to equilibrate the nsPAChA doses and the CRA doses in order to attain the maximum efficacy with reduced risk of both central and peripheral adverse effects, by using the nsPAChA at a daily dose which is from 0.5 to 8 times, advantageously from more than 1 to 8 times, preferably from 1.2 to 8 times, the daily dose of the brand or generic nsPAChA normally used in the anticholinergic therapy, concurrently with a high daily dose of CRA, in particular 1.2 to 4-times and even 1.2 to 6-times the maximum daily doses used in the clinical trials of said CRA.

### The CRAs

Any CRA which is able to cross the brain blood barrier of a human in order to stimulate the muscarinic cholinergic receptors in the CNS may be used as Component (b) according to the present invention.

Advantageously, the CRA used as Component (b) is one of the muscarinic cholinergic agonists that have extensively, but unsuccessfully been investigated in relation to the possibility of using them for the treatment of Alzheimer type dementia, as well as M₁ receptor positive allosteric modulators that are believed to be useful in the treatment of this and other diseases involving the muscarinic M₁ receptor.

Preferably, said CRA is selected from the group consisting of
1-methylpiperidine-4-spiro-5′(2′-ethyl-1′,4′-thiazoline-3′-one) (AF267) and pharmaceutically acceptable salts and solvates thereof, especially its hydrochloride (AF 267B) described in EP 0711292;
cis-2′-methylspiro {1-azabicyclo [2.2.2] octane-3,5′-[1,3] oxathiolane} described in U.S. Pat. No. 4,855,290 and U.S. Pat. No. 5,571,918 (cevimeline), and pharmaceutically acceptable salts and solvates thereof, especially its hydrochloride hemihydrate;
3-[3-(3-(3-fluorophenyl)-2-propyn-1l-ylthio)-1,2,5-thiadiazol-4-yl]-1,2,5,6-tetrahydro-1-methylpyridine described in CN 1821243B and pharmaceutically acceptable salts and solvates thereof, especially its oxalate (EUK 1001);
(E)-N-methoxy-1-(1 -methyl-1,2,5,6-tetrahydropyridin-3-yl)methanimine described in U.S. Pat. No. 6,037,347 (milameline) and pharmaceutically acceptable salts and solvates thereof, especially its hydrochloride;
2-ethyl-8-methyl-2,8-diazaspiro[4.5]decane-1,3-dione described in U.S. Pat. No. 3,056,796 (RS-86) and pharmaceutically acceptable salts and solvates thereof, especially its hydrobromide;
(3R)-N-methoxyquinuclidine-3-carboximidoyl cyanide described in U.S. Pat. No. 5,278,170 (sabcomeline) and pharmaceutically acceptable salts and solvates thereof, especially its hydrochloride;
(3R)-3-(prop-2-yn-1-yloxy)-1-azabicyclo[2.2.2]octane (talsaclidine) described in U.S. Pat. No. 5,286,864, and pharmaceutically acceptable salts and solvates thereof, especially its fumarate;
5-[4-(hexylthio)-1,2,5-thiadiazol-3-yl]-1-methyl-1,2,3,6-tetrahydropyridine described in U.S. Pat. No. 5,041,455 (tazomeline) and pharmaceutically acceptable salts and solvates thereof, especially its hydrochloride;
3-(4-hexyloxy-1,2,5-thiadiazol-3-yl)-1-methyl-5,6-dihydro-2H-pyridine described in U.S. Pat. No. 5,041,455 and EP 0384288 (xanomeline) and pharmaceutically acceptable salts and solvates thereof, especially its oxalate and L-tartrate,
(4-n-butyl-1-[4-(2-methylphenyl)-4-oxo-1-butyl]-piperidine (AC-42) and pharmaceutically acceptable salts and solvates thereof, especially its hydrogen chloride,
1-[1′-(2-methylbenzyl)-1,4′-bipiperidin-4-yl]-1,3-dihydro-2H-benzimidazol-2-one (TBPB) and pharmaceutically acceptable salts and solvates thereof;
4-Fluoro-6-methyl-1-[1-(tetrahydro-2H-pyran-4-yl)-4-piperidinyl]-1,3-dihydro-2H-benzimidazol-2-one and pharmaceutically acceptable salts and solvates thereof, described in WO 2007/036715;
5-Fluoro-6-methyl-1-[1-(tetrahydro-2H-pyran-4-yl)-4-piperidinyl]-1,3 -dihydro-2H-benzimidazol-2-one and pharmaceutically acceptable salts and solvates thereof; described in WO 2007/036718 and U.S. Pat. No. 8,288,412;
4-(R)-ethyl-3-(2-methylbenzamido)-1,4′ -bipiperidine-1′ -carboxylate and pharmaceutically acceptable salts and solvates thereof, described in WO 2010/096703;
ethyl 3-[(3-exo)-(2-benzamidoethyl)amino]-8-azabicyclo[3.2.1]octane-8-carboxylate and pharmaceutically acceptable salts and solvates thereof, described in U.S. Pat. No. 8,697691; and
3-[(1S,2S)-2-hydroxycyclohexyl]-6-[(6-methylpyridin-3-yl)methyl]benzo[h] quinazolin-4(3H)-one (MK-7622), described in U.S. Pat. No. 8,883,810 and pharmaceutically acceptable salts and solvates thereof; especially the fumarate or the hydrochloride.

The amount of the CRA Component (b) of the combination, i.e. a single CRA dose, may vary according to intrinsic muscarinic cholinergic receptor potency of said component. Advantageously, said dose is from 1.2-fold to 4-times and even from 1.2-fold to 6-times higher than the maximum amount contained in the commercial products or of the maximal, single CRA dose administered during the clinical trials of each CRA.

Thus, for example, cevimeline, as hydrochloride hemihydrates, is present in an amount of from 36 mg to 180 mg; milameline, as hydrochloride, is present in an amount of from 2.4 mg to 12 mg; xanomeline, as free base, as oxalate or as L-tartrate, is present in an amount of from 90 mg to 450 mg and MK-7622, especially as hydrochloride or fumarate, is present in an amount of from 6 mg to 54-270 mg, normally from 54 to 180 mg.

Advantageously, the daily dose of said CRA is higher than the average maximal tolerated dose of said CRA determined in its clinical trials. Preferably, it is from 1.2 to 4 times and even from 1.2 to 6 times said maximal tolerated CRA dose or from 1.2 to 4 times and even 1.2 to 6 times the maximal daily dose administered to patients during the clinical trials of each CRA.

In particular, the daily dose of cevimeline, as hydrochloride hemihydrate, is of from 108 mg to 180 mg and the daily dose of xanomeline, as oxalate or L-tartrate, is from more than 300 mg to 1350 mg, advantageously from 337.5 mg to 1350 mg, preferably from 337.5 to 900 mg and the dose of MK-7622 may be from 6 mg to 270 mg, advantageously from 54 mg to 270 mg, normally from 54 mg to 180 mg.

### The AChEIs

According to the present invention, the combination may contain, as a further component, Component (c) an AChEI also formulated in a pharmaceutical composition. Said AChEI may include, but is not limited to, 1,2,3,4-tetrahydro-9-acridinamine (tacrine) and pharmaceutically acceptable salts and solvates thereof, (1R,9S,13E)-1-amino-13-ethylidene-11-methyl-6-azatricyclo[7.3.1.0^{2,7}]trideca-2(7),3,10-trien-5-one (huperzine A, (±)-2,3-dihydro-5,6-dimethoxy-2-[[1-(phenylmethyl)-4-piperidinyl]methyl]-1H-inden-1-one (donepezil) and pharmaceutically acceptable salt and solvates thereof, (S)-N-Ethyl-N-methyl-3-[1-(dimethylamino)ethyl]-phenyl carbamate (rivastigmine) and pharmaceutically acceptable salts and solvates thereof, or 4aS,6R,8aS-3-methoxy-11-methyl-4a,5,9,10,11,12-hexahydroxy-6H-benzofuro[3a,3,2-e,f]benzazepin-6-ol (galantamine) and pharmaceutically acceptable salts and solvates thereof.

The AChEI Component (c) when included in the combination with Component (a), Component (b) as described herein, may be present in the amount currently used for treating Alzheimer disease, or also in a higher dose.

Said AChEIs may be used in brand preparation. For example, rivastigmine may be orally administeried by usind EXELON® immediate-release 3 mg or 6 mg-capsules or by applying EXELON® patches releasing 4.6 mg/24 hours, 9.5 mg/24 hours, or 13.3 mg/24 hours on the subject's skin.

Huperzine A may be used as a commercial preparation, by orally administering 0.05-0.2 mg immediate-release oral unit forms such as tablets or capsules.

Donepezil hydrochloride may be also used as a brand preparation, for example by orally administering ARICEPT® immediate-release 5 mg- or 10 mg-tablets or the 23 -mg tablets. In particular, donepezil hydrochloride may be orally administered, in combination with the above-illustrated MCRA and nsPAChA, at a daily dose preferably of from 5 mg to 60 mg.

Galantamine, as hydrobromide, may be also administered as a brand preparation, for example by orally administering RAZADYNE® immediate-release 8 mg- or 12 mg-tablets or RAZADYNE® ER 8 mg-, 16 mg- or 24 mg-capsules. In particular, galantamine hydrobromide may be orally administered, at a daily dose up to 42 mg.

Among the particularly preferred AChEIs, in the combinations of the present invention donepezil hydrochloride is present at a dose of from 5 mg to 60 mg, advantageously from 15 mg to 25 mg; rivastigmine, as hydrogen tartrate, is present, in a composition for oral administration, at a dose of from 3 mg to 15 mg, advantageously from 9 mg to 15 mg; as the free base, rivastigmine is present in patch releasing from 4.6 mg/24 h to 52 mg/24 h rivastigmine, advantageously from 9.6 mg/24 h to 33.25 mg/24 h, normally from 13.3 mg/24 h to 33.25 mg/24 h; and galantamine (as hydrobromide, is present in an amount of from 8 mg to 36 mg in an IR formulation or from 24 mg to 42 mg in an ER formulation.

### The Combinations

The present invention provides the combination of any nsPAChA and any CRA as illustrated in the respective above sections, each formulated in pharmaceutical composition in admixture with a pharmaceutical carrier.

According to an embodiment, an advantageous nsPAChA/CRA combination consists of

(a) any of the above-illustrated nsPAChAs, each in a pharmaceutical composition in admixture with a pharmaceutical carrier, said nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride and valethamate bromide; and
(b) cevimeline, as hydrochloride hemihydrate, in an amount of from 36 mg to 180 mg, in an IR-formulated oral composition in admixture with a pharmaceutical carrier.

A combination consisting of

(a) an nsPAChA selected from the group consisting of
anisotropine hydrobromide, in an amount of from 60 mg to 300 mg, normally from 60 mg to 200 mg in an IR-formulated oral composition in admixture with a pharmaceutical carrier;
glycopyrronium bromide in an amount of from 2.2 to 12 mg, normally from 2.2 to 8 mg in an IR-formulated oral composition in admixture with a pharmaceutical carrier;
butylscopolamine bromide in an amount of from 12 mg to 60 mg, normally from 12 mg to 40 mg in an IR-formulated oral composition in admixture with a pharmaceutical carrier;
otilonium bromide in an amount of from 48 mg to 240 mg, normally from 48 mg to 160 mg;
clidinium bromide in an amount of from 3 mg to 15 mg, normally from 3 mg to 12 mg in an IR-formulated oral composition in admixture with a pharmaceutical carrier;
prifinium bromide in an amount of from 36 mg to 180 mg, normally from 36 mg to 120 mg in an IR-formulated oral composition in admixture with a pharmaceutical carrier;
timepidium bromide in an amount of from 36 mg to 180 mg, normally from 36 mg to 120 mg in an IR-formulated oral composition in admixture with a pharmaceutical carrier; and
valethamate bromide in an amount of from 12 mg to 60 mg, normally from 12 mg to 40 mg in an IR-formulated oral composition in admixture with a pharmaceutical carrier;

in admixture with a pharmaceutical carrier in a composition formulated in an IR unit form; and

(b) cevimeline hydrochloride, in an amount of from 36 mg to 180 mg, in admixture with a pharmaceutical carrier in a composition formulated in an IR unit form,
is a preferred form of this embodiment.

According to another embodiment, an advantageous nsPAChA/CRA combination according to the present invention consists of

(a) any of the above-illustrated nsPAChAs, each in a pharmaceutical composition in admixture with a pharmaceutical carrier, said nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride and valethamate bromide; and
(b) xanomeline, as free base, as oxalate or as L-tartrate, in an amount of from 90 mg to 450 mg, normally from 90 to 300 mg, in an IR-formulated oral composition in admixture with a pharmaceutical carrier.

A combination consisting of

(a) an nsPAChA selected from the group consisting of
anisotropine hydrobromide, in an amount of from 60 mg to 300 mg, normally from 60 mg to 200 mg in an IR-formulated composition;
glycopyrronium bromide in an amount of from 2.2 to 12 mg, normally from 2.2 to 8 mg in an IR-formulated oral composition in admixture with a pharmaceutical carrier;
butylscopolamine bromide in an amount of from 12 mg to 60 mg, normally from 12 mg to 40 mg in an IR-formulated oral composition in admixture with a pharmaceutical carrier;
otilonium bromide in an amount of from 48 mg to 240 mg, normally from 48 mg to 160 mg in an IR-formulated oral composition in admixture with a pharmaceutical carrier;
clidinium bromide in an amount of from 3 mg to 15 mg, normally from 3 mg to 12 mg in an IR-formulated oral composition in admixture with a pharmaceutical carrier;
prifinium bromide in an amount of from 36 mg to 180 mg, normally from 36 mg to 120 mg in an IR-formulated oral composition in admixture with a pharmaceutical carrier;
timepidium bromide in an amount of from 36 mg to 180 mg, normally from 36 mg to 120 mg in an IR-formulated oral composition in admixture with a pharmaceutical carrier; and
valethamate bromide in an amount of from 12 mg to 60 mg, normally from 12 mg to 40 mg in an IR-formulated oral composition in admixture with a pharmaceutical carrier; and
   (b) xanomeline, as free base, as oxalate or as L-tartrate, in an amount of from 90 mg to 450 mg, normally from 90 to 300 mg, in an IR-formulated oral composition in admixture with a pharmaceutical carrier,
   is a preferred form of this embodiment.

According to another embodiment, an advantageous nsPAChA/CRA combination according to the present invention consists of

(a) any of the above-illustrated nsPAChAs, each in a pharmaceutical composition in admixture with a pharmaceutical carrier, said nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride and valethamate bromide; and
(b) milameline hydrochloride, in an amount of from 2.4 mg to 12 mg, normally from 2.4 to 10 mg, in an IR-formulated oral composition in admixture with a pharmaceutical carrier.

According to another embodiment, an advantageous nsPAChA/CRA combination according to the present invention consists of

(a) any of the above-illustrated nsPAChAs, each in a pharmaceutical composition in admixture with a pharmaceutical carrier, said nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride and valethamate bromide; and
(b) MK-7622, as free base, as hydrochloride or as fumarate, in an amount of from 6 mg to 270 mg, normally from 4 to 180 mg, in an IR-formulated oral composition in admixture with a pharmaceutical carrier.

A combination consisting of

(a) an nsPAChA selected from the group consisting of
fesoterodine fumarate, in an amount of from 9.6 mg to 32 mg, in an ER-formulated oral composition in admixture with a pharmaceutical carrier;
propiverine hydrochloride, in an amount of from 36 mg to 120 mg, in an ER-formulated oral composition in admixture with a pharmaceutical carrier;
solifenacin succinate, in an amount of from 12 mg to 40 mg, normally from 12 mg to 20 mg, in an IR-formulated oral composition in admixture with a pharmaceutical carrier;
tolterodine tartrate, in an amount of from 4.8 mg to 16 mg, in an IR-formulated oral composition in admixture with a pharmaceutical carrier;
trospium chloride ER, in an amount of from 72 mg to 240 mg in an ER-formulated oral composition in admixture with a pharmaceutical carrier; and
   (b) MK-7622, as free base, as hydrochloride or as fumarate, in an amount of from 6 mg to 270 mg, normally from 4 to 180 mg, in an IR-formulated oral composition in admixture with a pharmaceutical carrier,
   is a preferred form of this embodiment.

### The Fixed-Dose Combination

As indicated above, the pharmaceutical compositions prepared by using the nsPAChAs according to the present invention are present in unit forms also containing a CRA that acts as direct cholinergic agent in the CNS to improve the symptoms of Alzheimer type dementia.

Thus, it is another object of the present invention to provide a pharmaceutical unit form that comprises

(a) a muscarinic receptor antagonist selected from the group consisting of the non- selective, peripheral anticholinergic agents (nsPAChAs); and
(b) a muscarinic receptor agonist selected from the group consisting of cholinergic receptor agonists (CRA),
in admixture with at least a pharmaceutical carrier.

The pharmaceutical composition to improve the treatment of human dementias of the Alzheimer type according to the present invention may comprise a mixture of a nsPAChA [Component (a)] and of a CRA [Component (b)], wherein Component (b) is present in a quantity sufficient to maximally alleviate disease-associated neurobehavioral symptoms and wherein Component (a), which does not appreciably penetrate the blood brain barrier, is present in a second quantity that reduces peripherally mediated adverse effects that would be caused by the CRA if administered without the accompanying nsPAChA.

Advantageous nsPAChAs are solifenacin and its salts, propiverine and its salts, oxyphencyclimine and its salts, tolterodine and its salts, fesoterodine and its salts; and quaternary ammonium salts or sulfonium salts of formula I above, such as homatropine quaternary salts, anisotropine quaternary salts, trospium quaternary salts, clidinium quaternary salts, benzilonium quaternary salts and glycopyrronium quaternary salts. Other suitable quaternary ammonium salts are scopolamine methobromide, scopolamine butylbromide, scopolamine methonitrate, isopropamide iodide, valethamate bromide, atropine methobromide, atropine methonitrate, diponium bromide, pipenzolate bromide, penthienate bromide, benactizine methobromide, diphemanil, emeprioum bromide and dibutoline sulfate.

Anisotropine hydrobromide; butylscopolamine bromide; cimetropium bromide; clidinium bromide; glycopyrronium bromide; methylpropiverinium iodide or bromide; otilonium bromide; prifinium bromide; timepidium bromide; trospium chloride, succinate, maleate, fumarate or tartrate; valethamate bromide; fesoterodine and its fumarate; oxyphencyclimine and its hydrochloride; propiverine and its hydrochloride; solifenacin and its succinate; tolterodine and the L-hydrogen tartrate thereof are particularly advantageous nsPAChAs used as Component (a).

Advantageous components (b) are the aforementioned CRA, in particular AF267 and pharmaceutically acceptable salts and solvates thereof, especially its hydrochloride (AF 267B), 3-[(1S ,2S)-2-hydroxycyclohexyl]-6-[(6-methylpyridin-3-yl)methyl]benzo[h] quinazolin-4(3H)-one (MK-7622) and pharmaceutically acceptable salts and solvates thereof, especially its hydrochlorideor fumarate, 3-[3-(3-(3-fluorophenyl)-2-propyn-1-ylthio)-1,2,5-thiadiazol-4-yl]-1,2,5,6-tetrahydro-1-methylpyridine (EUK 1001) and pharmaceutically acceptable salts and solvates thereof especially its oxalate, milameline and pharmaceutically acceptable salts and solvates thereof especially its hydrochloride, RS-86 and pharmaceutically acceptable salts and solvates thereof, especially its hydrobromide; sabcomeline and pharmaceutically acceptable salts and solvates thereof; talsaclidine and pharmaceutically acceptable salts and solvates thereof, especially its fumarate; tazomeline and pharmaceutically acceptable salts and solvates thereof, especially its hydrochloride; xanomeline and pharmaceutically acceptable salts and solvates thereof, especially its oxalate and its L-tartrate.

Particularly advantageous Component (b) is a CRA selected from the group consisting of cevimeline and pharmaceutically acceptable salts thereof, milameline and pharmaceutically acceptable salts and solvates thereof, xanomeline and pharmaceutically acceptable salt and solvates thereof. Cevimeline, its hydrochloride hemihydrate, xanomeline and its oxalate or L-tartrate are the preferred Components (b).

The dose of the Component (b) may vary according to the intrinsic muscarinic cholinergic potency and to the administration route of said component. Advantageously, said dose is from 1.4-fold to 4 times, or 1.2-fold to 6-times higher than the mean maximal tolerated dose determined in the clinical trials

In the unit forms of the present invention, for immediate release or extended release, the nsPAChA Component (a) is present in an amount of from 50% to 600%, preferably from 1.2-fold to 6 times the maximum IR amount of said nsPAChA contained in the currently administered IR dosage unit forms for the treatment of disorders such as gastrointestinal cramps, urinary bladder spasm, asthma, motion sickness, muscular spasms and the CRA Component (b) is present in an amount of from 100% to 600, preferably from 120% to 600%, the maximum amount of said CRA contained in the IR dosage unit forms administered for the approved indication or in the clinical trials.

More particularly, the nsPAChA is present, in an IR unit form, in an amount ranging from 50% to 400%, preferably from 120% to 400%, the maximum amount of said nsPAChA contained in the currently administered IR dosage unit forms for the treatment of the above-cited disorders or, in an ER unit form, in an amount ranging from 75% to 600%, preferably from 120% to 600%, the maximum amount of said nsPAChA contained in the currently administered unit dosage IR forms for the treatment of the above-cited disorders.

For example, among the nsPAChAs used as Component (a),
anisotropine hydrobromide is present in an amount of from 120 mg to 400 mg;
butylscopolamine bromide is present in an amount of from 12 mg to 40 mg;
cimetropium bromide is present in an amount of from 55 mg to 200 mg;
clidinium bromide is present in an amount of from 3 mg to 10 mg;
fesoterodine fumarate ER is present in an amount of from 9.6 mg to 32 mg;
glycopyrronium bromide is present in an amount of from 2.4 mg to 8 mg;
otilonium bromide is present in an amount of from 48 mg to 160 mg;
oxyphencyclimine is present in an amount of from 18 mg to 60 mg;
prifinium bromide is present in an amount of from 36 mg to 120 mg;
propiverine hydrochloride IR is present in an amount of from 18 mg to 60 mg;
propiverine hydrochloride ER is present in an amount of from 36 mg to 120 mg;
solifenacin succinate is present in an amount of from 12 mg to 40 mg;
tolterodine hydrogen tartrate is present in an amount of from 4.8 mg to 16 mg;
timepidium bromide is present in an amount of from 36 mg to 120 mg;
trospium chloride IR is present in an amount of from 24 mg to 80 mg;
trospium chloride ER is present in n an amount of from 72 mg to 240 mg; and
valethamate bromide is present in an amount of from 12 mg to 40 mg.

In unit form for immediate release or extended release, the CRA Component (b) is present in an amount of from about 100% to about 600%, advantageously from 120% to 600% or 150% to 600% the maximum amount of said CRA contained in the commercial IR unit forms or the maximum tolerated amount of a single unit forms used in the clinical trials. Normally, the CRA Component (b) is present, in an IR-form, in an amount of from 100% to 400%, preferably from 120% to 400%, the CRA maximum amount contained in the commercial IR unit forms or the maximum tolerated amount of a single IR unit forms used in the clinical trials; and in an ER-form in an amount of from 120% to 600%, preferably from 150% to 600%, the CRA maximum amount contained in the commercial IR unit forms or the maximum tolerated amount of a single IR unit forms used in the clinical trials.

For example, among the preferred Components (b),
cevimeline is present, as hydrochloride hemihydrate, in an amount of from 30 mg to 120 mg, preferably from 36 mg to 120 mg in an oral IR or, as free base or as hydrochloride hemihydrate, from 36 mg to 180 mg, preferably from 45 mg to 180 mg in an oral or transdermal ER form, in particular in a TTS;
milameline is present, as hydrochloride, in an amount of from 2 mg to 8 mg, preferably from 2.4 mg to 8 mg in an oral IR or, as free base or as hydrochloride, from 2.4 mg to 12 mg, preferably from 3 mg to 12 mg in an oral or transdermal ER form, in particular in a TTS; and
xanomeline is present, as oxalate or L-tartrate, in an amount of from 75 mg to 300 mg preferably from 90 mg to 300 mg in an oral IR form or, as free base, as oxalate or as L-tartrate, from 90 mg to 450 mg, preferably from 112.5 mg to 450 mg in an oral or transdermal ER form, in particular in a TTS
MK-7622 is present, as hydrochloride or fumarate, in an amount of from 6 mg to 270 mg preferably from 54 mg to 180 mg in an oral IR form or, as free base, as fumarate or as hydrochloride, from 54 mg to 270 mg, preferably from 54 mg to 180 mg in an oral or transdermal ER form, in particular in a TTS.

Any nsPAChA and any CRA as illustrated in the above “The Combinations” section may be formulated in a pharmaceutical composition in a single unit form, in admixture with at least one pharmaceutical carrier according the “The Formulations” section below.

A preferred CRA/nsPAChA fixed-dose combination essentially consists of a pharmaceutical composition in dosage unit form comprising

(a) a CRA selected from the group consisting of MK-7622 and pharmaceutically acceptable salts thereof, especially its fumarate, methanesulfonate or hydrochloride, in an amount of from 6 mf to 270 mg; and
(b) a nsPAChA selected from the group consisting of solifenacin and pharmaceutically acceptable salts thereof, in particular its succinate, in an amount corresponding to from 10 mg to 80 mg, preferably from 10 mg to 40 mg of solifenacin succinate,
in admixture with a pharmaceutical carrier.

According to an embodiment, the fixed-dose combination may comprise an AChEI component (c), for example donepezil and pharmaceutically acceptable salts thereof, especially its hydrochloride; rivastigmine and pharmaceutically acceptable salts thereof, especially its hydrogen tartrate; galantamine and pharmaceutically acceptable salts thereof, especially its hydrobromide; huperzine A, at a dose among those described in the above “The AChEIs” section,

A preferred CRA/nsPAChA/AChEI fixed-dose combination essentially consists of a pharmaceutical composition in dosage unit form comprising

(a) a CRA selected from the group consisting of MK-7622 and pharmaceutically acceptable salts thereof, especially its fumarate, methanesulfonate or hydrochloride, in an amount of from 6 mf to 270 mg;
(b) a nsPAChA selected from the group consisting of solifenacin and pharmaceutically acceptable salts thereof, in particular its succinate, in an amount corresponding to from 10 mg to 80 mg, preferably from 10 mg to 40 mg of solifenacin succinate, and
(c) an AChEI selected from the group consisting of donepezil and pharmaceutically acceptable salts thereof, especially its hydrochloride, in an amount of from 5 mg to 60 mg;
in admixture with a pharmaceutical carrier.

### The Formulations

The unit form of the present invention may be a tablet, a capsule, a pre-measured volume of a liquid solution or suspension for oral administration or a TTS as a gel or patch for transdermal application. In said unit form the nsPAChA and the CRA, as free base are as a pharmaceutically acceptable salt or solvate thereof, may be mixed together or separated according to known technologies in admixture with a pharmaceutical carrier in a pharmaceutical composition.

Component (a) and Component (b) are formulated with conventional pharmaceutical carriers in known formulations for oral use wherein said components are mixed together or separated, for example in two tablets introduced in a capsule or in a two-compartment capsule or in a multilayer (di-layer) tablet wherein the two components are both in IR or in ER form or one of the two components is in IR form and the other is in ER form, according to known technologies.

The pharmaceutical carriers and vehicles are those commonly used for the preparation of compositions for oral, buccal and parenteral, in particular transdermal, administration. Appropriate unit forms comprise the oral forms such as tablets, soft or hard gelatin capsules, powders or granulates in sachets and suitably measured oral solutions or suspensions as well as patches for transdermal administration.

Component (a) and Component (b) may also be present in form of one of their complexes with a cyclodextrin, for example α-cyclodextrin, β-cyclodextrin, γ-cyclodextrin, 2-hydroxypropyl-β-cyclodextrin or methyl-β-cyclodextrin.

Component (a) and Component (b) may also be formulated in the form of microcapsules, optionally with one or more carriers or additives.

For oral administration, Component (a) and Component (b), together or separately, are formulated by mixing the active ingredient with conventional pharmaceutical acceptable carriers enabling said active ingredients to be formulated in tablets, dragees, orally disintegrating tablets, capsules, liquid solutions or suspensions, syrups and the like.

Carriers for IR tablets include for example starches, cellulose and derivatives thereof; lubricants such as talc, stearic acid or magnesium stearate; diluents such as talc, powdered cellulose, lactose, starches such as maize or corn starch, mannitol, sorbitol; disaggregating agents such as microcrystalline cellulose or crospovidone; lubricants such as polyethylene glycol or magnesium stearate; ligands such as methylcellulose, sodium carboxymethylcellulose, alginic acid, alginates; sweeteners, such as sucrose, dextrose, mannitol, saccharin; or flavoring agents such as natural or synthetic oils.

Carriers for orally disintegrating tablets include for example lubricants, aggregating, sweetening, flavoring or disaggregating agents as well as agents improving the buccal mucosa absorption of Components (a) and (b) such as sorbitol, mannitol, lactose and cellulose.

Carriers for liquid, normally aqueous, suspensions or solutions include for example antioxidants, such as sodium metabisulfite or sodium sulfite, thickening agents, such as microcrystalline cellulose, hydroxypropylcellulose, carboxymethylcellulose or polyvinylpyrrolidone, preservatives such as methyl paraben, ethyl paraben, sodium ethylenediaminotetracetate, sodium benzoate or an alkaline salt of sorbic acid, as well as flavoring and sweetening agents.

The sweeteners contained in the orally disintegrating tablets and the liquid suspensions or solutions may be natural, optional reduced sugars such as sucrose, dextrose, xylitol, mannitol or sorbitol, or synthetic product such as sodium saccharine or aspartame.

The flavoring agents are pharmaceutically acceptable flavors and tastes of synthetic and natural oils, the latter extracted from plants, leaves, flowers, fruits and their combinations, such as cinnamon, peppermint, anise and citron leaves, bitter almond, citrus fruits, in particular orange and/or lemon, linden and grapefruit oils. Also chocolate, vanilla or eucalyptus flavor and essences of fruit, in particular apple, pear, peach, strawberry, cherry, apricot, orange, lemon and grapes may be advantageously used. The composition according to the present invention may be in form of a capsule containing two tablets as described herein above, one of them comprising Component (a) and the other comprising Component (b).

The combination may be formulated in tablets in which one or both of the two components is in controlled-release formulation, for example as a dispersion of said component in hydroxypropyl methyl cellulose or in a film-coated microgranule.

Advantageously, the CRA, in an ER-formulation is in the core and the nsPAChA, in IR-formulation, is in the outer layer in multi-layer tablets in which, for example, both the core and the outer layer are coated with a film. Analogously, capsules made of two separated parts, one containing Component (a), in IR- or ER-formulation and the other containing Component (b), in IR- or ER-formulation, may be used.

Carriers and vehicles for ER tablets include retardant materials such as acrylic and methacrylic acid polymers and copolymers; cellulose derivatives such as hydroxypropylmethylcellulose, hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

Component (a) and Component (b), as the base thereof or as a pharmaceutically acceptable salt thereof, may also be formulated in a delivering transdermal pharmaceutical form, such as a patch, a gel, a cream, a spray, an ointment, a lotion or a paste, wherein Component (a), Component (b) or both the Components (a) and (b) are present in admixture with the common diluents and permeation enhancers.

The permeation enhancer may be any compound which allows the improved permeation of drugs through the skin (see for example the review in Pharmaceutical Technology, November 1997, pages 58-66, the disclosure of which is herein incorporated by reference in its entirety). Such substances may be lower (C₁-C₄) alkanols; fatty alcohols such as lauryl alcohol (dodecanol), alone or in combination with a lower alkanol; fatty acids such as linolenic acid or oleic acid; fatty acid esters such as isopropyl palmitate, stearate, linoleate, oleate or myristate; glycerol; glycerol monoesters such as glycerol monostearate, monolinoleate or monooleate; glycerol diesters; glycerol triesters such as triacetin; sucrose monostearate, monolinoleate or monooleate; sorbitan esters; fatty alcohol ethers having from 10 to 20 carbon atoms; glycols, such as diethylene glycol or propylene glycol; glycols lower alkyl ethers, such as diethylene glycol mono(C₂-C₄)alkyl ether, in particular diethylene glycol monoethyl ether.

These permeation enhancers are present in an amount from 0.01 to 20% by weight of the total weight of the composition, advantageously in an amount of from 0.05 to 10% by weight, preferably from 0.1 to 5% by weight.

### The Use

As set forth herein above, Component (a) and Component (b) may be administered concurrently or sequentially to a patient suffering from Alzheimer type dementia. In particular, Component (a) and Component (b) can be administered in a specific dosage regimen to treat Alzheimer type dementia, Component (a) and Component (b) being administered simultaneously or sequentially to one another, in each case by the same or different administration route.

Component (a) and Component (b) may also be present in the same unit form, each in the dose, on one side—Component (a)—allowing the safe administration of event high doses of Component (b) without the dangerous adverse effects linked to the peripheral cholinergic action of said Component (b); and, on the other side—Component (b)—capable of safely improving cognition of patients suffering from Alzheimer type dementia, thanks to peripheral anticholinergic action of Component (a).

Thus, according to another of its aspects, the present invention provides a combination comprising, as Components:

(a) a muscarinic receptor antagonist selected from the group consisting of the non-selective, peripheral anticholinergic agents (nsPAChAs); and
(b) a muscarinic receptor agonist selected from the group consisting of cholinergic receptor agonists (CRA),
for use in the treatment of Alzheimer type dementia.

The nsPAChA used as Component (a), their properties and doses are described in the “nsPAChAs” section above.

The CRAs used as Component (b), their properties and doses are described in the “CRAs” section above.

For the use, Component (a) and Component (b), together or separately, are formulated in pharmaceutical compositions prepared as described in the “Formulation” section above.

According to another of its aspects, the present invention provides a method for treating Alzheimer type dementia, which comprises administering to a patient in need of said treatment a combination comprising, as Components:

(a) a muscarinic receptor antagonist selected from the group consisting of the non-selective, peripheral anticholinergic agents (nsPAChAs); and
(b) a muscarinic receptor agonist selected from the group consisting of cholinergic receptor agonists (CRA).

The method is carried out by administering Component (a) and Component (b) of said combination concurrently, or sequentially. Component (a) and Component (b) may be independently administered by oral or parenteral route, in particular by intramuscular or intravenous injection or by transdermal administration by a TTS such as a gel or a patch.

The nsPAChA used as Component (a), their properties and doses are described in the “nsPAChAs” section above.

The CRAs used as Component (b), their properties and doses are described in the “CRAs” section above.

For administering the combination to said patient, Component (a) and Component (b), together or separately, are formulated in pharmaceutical compositions prepared as described in the “Formulation” section above.

In the case of simultaneous administration of the two components, Component (a) and Component (b) may be associated in the same pharmaceutical composition, in a unit dose for oral or parenteral, including transdermal, route.

The following examples are included for illustrative purposes only, and are not intended to limit the scope of the invention.

### EXAMPLE 1

### Study 1—Establishment of the Dose-Response to Xanomeline in a Mouse Model of Diarrhea.

Male Swiss mice (4-6 weeks old), N=10 per treatment group were used, and treated intra-peritoneally (i.p.) with either vehicle (vehicle group) or increasing doses of xanomeline, a representative muscarinic agonist. Mice were randomly assigned to one of two experimental groups (vehicle; or increasing doses of xanomeline). Each animal was identified by its group name, cage number, series (day) of experiment, and number (1 to 10) written with permanent ink on the tail.
Mice were placed individually in cages without any bedding materials. During the experiment the number of fecal pellets were counted at different time-points, starting one hour before the time of the administration of the test compound (TO), as outlined below:
T-1 h to T0: counting of the accumulated fecal pellets excreted.
T0: administration of the test compound,
T0 to T+2 h: counting of the accumulated fecal pellets excreted,
T+2 h to T+4 h: counting of the accumulated fecal pellets excreted,
   The total number of fecal pellets for each mouse was counted over time. An analysis of variance (ANOVA) was performed on the results. Fisher's Protected Least Significant Difference was used for pairwise comparisons; p values <0.05 were considered significant. Grubbs' test (http (hypertext transfer protocol) www at graphpad.com/quickcalcs/Grubbs1.cfm) was used to detect outliers for each parameter in each experimental group.
   Results confirmed that xanomeline i.p. (0.3 to 30 mg/kg) dose-dependently causes diarrhea.

### Study 2 - Antagonism of Xanomeline-Induced Diarrhea in Mice by Oxybutynin

Male Swiss mice (4-6 weeks old), N=10 per treatment group were used. Animals were pretreated with i.p. oxybutynin (a representative peripheral muscarinic receptor antagonist) or vehicle; 30 minutes later animals were treated with xanomeline at a dose of 30 mg/kg that caused diarrhea (as determined in Experiment 1). The dose of oxybutynin ordinarily ranged from 0.3 to 30 mg/kg. Mice were placed individually in cages without any bedding materials. During the experiment the number of fecal pellets was counted at different time-points as outlined below:
T-1h to T0: counting of the accumulated fecal pellets excreted,
T0: administration of oxybutinin.
T30 min: administration of vehicle or xanomeline.
T 30 min to T 2.5 h: counting of accumulated fecal pellets excreted.
T+2.5 h to T+4.5 h: counting of accumulated fecal pellets excreted.
   The total number of fecal pellets for each mouse was counted over time. An analysis of variance (ANOVA) was performed on the results. Fisher's Protected Least Significant Difference was used for pairwise comparisons. The p value ≦0.05 were considered significant. Grubbs' test (http (hypertext transfer protocol) www at graphpad.com/quickcalcs/Grubbs1.cfm) was used to detect outliers for each parameter in each experimental group.
   Results showed that oxybutynin dose-dependently antagonized the diarrhea induced by xanomeline, thus confirming that the representative nsPAChA oxybutynin suppresses the adverse effects of the representative muscarinic antagonist xanomeline.

### EXAMPLE 2

Evaluation of Cognition with Oxybutynin and Xanomeline in the T-maze Alternation Task in Mice
The T-maze continuous alternation task (T-CAT) is useful as model for studying compounds with cognitive enhancing properties. The T-maze consists of 2 choice arms and 1 start arm mounted to a square center. Manual doors are provided to close specific arms during the force choice alternation task.
Male Swiss mice (4-6 weeks old), N=10 per treatment group were used, and were pre-treated with:
Oxybutynin at the dose that blocked fecal pellet excretion in Study 2 of Example 1. Thirty minutes later mice were treated with either vehicle or one of 4 doses of xanomeline:
the highest dose that did not cause diarrhea;
a dose that caused diarrhea.
   Mice were randomly assigned to one of the different experimental treatment groups. Each animal was identified by its group name, cage number, series (day) of experiment, and number (1 to 10) written with permanent ink on the tail.
   The T-maze apparatus is made of gray Plexiglas with a main stem (55 cm long×10 cm wide×20 cm high) and two arms (30 cm long×10 cm wide×20 cm high) positioned at 90 degree angle relative to the main stem. A start box (15 cm long×10 cm wide) is separated from the main stem by a guillotine door. Horizontal doors are also provided to close specific arms during the force choice alternation task.

The experimental protocol consisted of one single session, which started with 1 “forced-choice” trial, followed by 14 “free-choice” trials. In the first “forced-choice” trial, animals were confined for 5 seconds to the start arm and then were released while either the left or the right goal arm was blocked by the horizontal door. Animals then negotiated the maze, eventually entering the open goal arm, and returned to the start position. Immediately after the return of the animals to the start position, the left or right goal door was opened and the animals were allowed to choose freely between the left and right goal arm (“free choice trials). An animal was considered as having entered in arm when it placed its four paws in the arm. A session was terminated and animals were removed from the maze as soon as 14 free-choice trials had been performed or 10 min had elapsed, whichever event occurred first.

The apparatus was cleaned between each animal using 40% ethanol. Urine and feces were removed from the maze. During the trials, animal handling and the visibility of the operator was minimized as much as possible.
The percentage of alternation over the 14 free-choice trials was determined for each mouse and was used as an index of working memory performance. This percentage is defined as entry in a different arm of the T-maze over successive trials (i.e., left-right-left-right, etc).
Analysis of variance (ANOVA) was performed on the results. Fisher's Protected Least Significant Difference was used for pairwise comparisons; p values ≦0.05 were considered significant. The drug-induced improvement of memory was calculated by setting the respective response of the saline/vehicle as 100% and that of the test group as 0% reversion. Grubbs' test (http (hypertext transfer protocol) www at graphpad.com/quickcalcs/Grubbs1.cfm) was used to detect outliers for each parameter in each experimental group.
Results showed a dose-dependent increase in performance in the T-maze in animals treated with i.p. xanomeline. At the higher dose, however, animals were too sick to perform the test. Pretreatment with i.p. oxybutynin restored the animals' ability to perform the T-maze test.

1. A pharmaceutical combination comprising as Components:
(a) a muscarinic receptor antagonist selected from the group consisting of the non-selective, peripheral anticholinergic agents (nsPAChAs); and
(b) a muscarinic receptor agonist selected from the group consisting of cholinergic receptor agonists (CRA).

2. The combination of claim 1, wherein said muscarinic receptor antagonist is an nsPAChA selected from the group consisting of quaternary ammonium nsPAChAs, sulfonium nsPAChAs, (1S)-(3R)-1-azabicyclo[2.2.2]oct-3-yl3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)-N,N-diisopropyl-3-(2-hydroxy-5-methylphenyl)-3-phenylpropanamine (tolterodine) and its pharmaceutically acceptable salts, [2-[(1R)-3-(Di(propan-2-yl)amino)-1-phenylpropyl]-4-(hydroxymethyl)phenyl] 2-methylpropanoate (fesoterodine) and its pharmaceutically acceptable salts.

3. The combination of claim 2 wherein said quaternary ammonium nsPAChAs or sulfonium nsPAChAs has the formula (I) 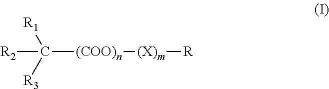
wherein
R is a radical selected from the group consisting of those of formulas (a)-(e) 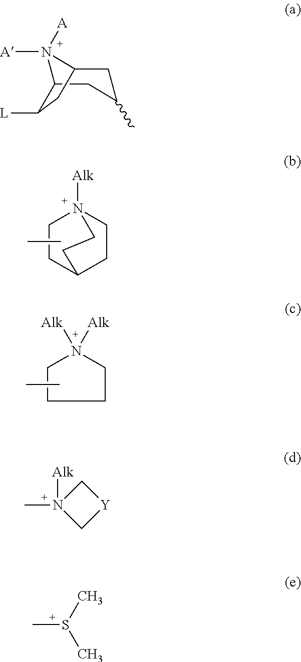
A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group.

4. The combination of claim 1, wherein said muscarinic receptor antagonist is a nsPAChA selected from the group consisting of azoniaspiro[3β-benziloyloxy-(1α,5α)-nortropane-8,1′-pyrrolidine] (trospium) chloride, 3-[2-cyclopentyl(hydroxy)phenylacetoxy]-1,1-dimethylpyrrolidinium (glycopyrronium) bromide, solifenacin and the compound thereof with succinic acid (solifenacin succinate), propiverine and the hydrochloride thereof, oxyphencyclimine and the hydrochloride thereof, tolterodine and the hydrogen tartrate thereof, fesoterodine and the fumarate thereof.

5. The combination of claim 1, wherein said muscarinic receptor agonist is a CRA selected from the group consisting of 1-methylpiperidine-4-spiro-5′(2′-ethyl-1′,4′-thiazoline-3′-one) (AF267) and pharmaceutically acceptable salts and solvates thereof; cis-2′-methylspiro {1-azabicyclo [2.2.2] octane-3,5′-[1,3] oxathiolane} (cevimeline) and pharmaceutically acceptable salts and solvates thereof; 3-[3-(3-(3-fluorophenyl)-2-propyn-1-ylthio)-1,2,5-thiadiazol-4-yl]-1,2,5,6-tetrahydro-1-methylpyridine and pharmaceutically acceptable salts and solvates thereof; (E)-N-methoxy-1-(1-methyl-1,2,5,6-tetrahydropyridin-3-yl)methanimine (milameline) and pharmaceutically acceptable salts and solvates thereof; 2-ethyl-8-methyl-2,8-diazaspiro[4.5] decane-1,3-dione (RS-86) and pharmaceutically acceptable salts and solvates thereof; (3R)-N-methoxyquinuclidine-3-carboximidoyl cyanide (sabcomeline) and pharmaceutically acceptable salts and solvates thereof; (3R)-3-(prop-2-yn-1-yloxy)-1-azabicyclo[2.2.2]octane (talsaclidine) and pharmaceutically acceptable salts and solvates thereof; 5-[4-(hexylsulfanyl)-1,2,5-thiadiazol-3-yl]-1-methyl-1,2,3,6-tetrahydropyridine and pharmaceutically acceptable salts and solvates thereof; 3-(4-hexyloxy-1,2,5-thiadiazol-3-yl)-1-methyl-5,6-dihydro-2H-pyridine (xanomeline), 3-[(1S,2S)-2-hydroxycyclohexyl]-6-[(6-methylpyridin-3-yl)methyl]benzo[h] quinazolin-4(3H)-one (MK-7622) and pharmaceutically acceptable salts and solvates thereof.

6. The combination according to claim 1 wherein said nsPAChA is formulated in a pharmaceutical composition or device in admixture with a pharmaceutical carrier or vehicle.

7. The combination of claim 6, wherein said composition or device further comprises the a CRA of claim 5.

8. The combination of claim 6, wherein said pharmaceutical composition or device comprises said nsPAChA at a dose from 50% to 400% the maximal dose contained in a corresponding marketed composition.

9. The composition of claim 8, wherein said composition or device further comprises a CRA.

10. The combination of claim 9, wherein said CRA is cevimeline hydrochloride hemihydrate or xanomeline hydrochloride.

11. The combination of claim 10, wherein said CRA is cevimeline hydrochloride hemihydrate, in an amount of from 34.5 mg to 180 mg.

12. The combination according to claim 1, wherein said CRA is MK-7622 hydrochloride or MK-7622 fumarate, in an amount of from 6 mg to 270 mg.

13. The combination according to claim 1, also comprising an AChEI selected from the group consisting of 1,2,3,4-tetrahydro-9-acridinamine (tacrine) and pharmaceutically acceptable salts and solvates thereof, (±)-2,3-dihydro-5,6-dimethoxy-2-[[1-(phenylmethyl)-4-piperidinyl]methyl]-1H-inden-1-one (donepezil) and pharmaceutically acceptable salt and solvates thereof, (S)-N-Ethyl-N-methyl-3-[1-(dimethylamino)ethyl]phenyl carbamate (rivastigmine) and pharmaceutically acceptable salts and solvates thereof, 4aS,6R,8aS-3-methoxy-11-methyl-4a,5,9,10,11,12-hexahydroxy-6H-benzofuro[3a,3,2-e,f]benzazepin-6-ol (galantamine) and pharmaceutically acceptable salts and solvates thereof.

14. The combination of claim 13, wherein said AChEI is in an amount selected from the group consisting of from 10 to 60 mg donepezil hydrochloride; from 18 to 48 mg rivastigmine, as hydrogen tartrate, and from 36 to 72 mg galantamine, as hydrobromide.

15. The combination according to claim 1, wherein said CRA and said nsPAChA are in a fixed-dose combination.

16. The combination of claim 15, wherein said fixed-dose combination consists of a pharmaceutical composition in dosage unit form comprising
(a) a CRA selected from the group consisting of MK-7622 and pharmaceutically acceptable salts thereof, especially its fumarate, methanesulfonate or hydrochloride, in an amount of from 6 mf to 270 mg; and
(b) a nsPAChA selected from the group consisting of solifenacin and pharmaceutically acceptable salts thereof, in particular its succinate, in an amount corresponding to from 10 mg to 80 mg of solifenacin succinate,
in admixture with a pharmaceutical carrier.

17. The composition of claim 15, wherein said fixed-dose combination is formulated for oral, intramuscular, intravenous, subcutaneous, intradermal, transdermal, transmucosal, intranasal, or rectal administration.

18. The combination or composition according to claim 1, for use in the treatment of hypocholinergic disorders.

19. A method for treating hypocholinergic disorders of the central nervous system, which comprises administering, to a patient in need of such a treatment, the combination according to claim 1.

20. The method of claim 18, wherein Component (a) and Component (b) of the combination are administered concurrently or sequentially to a patient suffering from a hypocholinergic disorder of the central nervous system, each Component being administered to said patient by the same or by a different administration route.

21. The method of claim 21, wherein said hypocholinergic disorder of the central nervous system is selected from the group consisting of Alzheimer disease, Alzheimer-type dementia, mild cognitive impairment, Lewy body disease dementia, Parkinson's disease dementia, post-stroke dementia, vascular dementia, traumatic brain injury, Down syndrome, anorexia nervosa, Tourette disease, tardive dyskinesia, Pick's disease, Huntington's chorea, Friedrich's ataxia, chronic neuropathic pain and schizophrenia.
